(19) United States
(12) Patent Application Publication
(10) Pub. No.: US 2003/0208231 A1
(43) Pub. Date: Nov. 6, 2003

(54) Surgical clamp devices and methods especially useful in cardiac surgery

(76) Inventors: Warren P. Williamson IV, Loveland, OH (US); Paul A. Spence, Louisville, KY (US); Craig B. Berky, Milford, OH (US)

Correspondence Address: WOOD, HERRON & EVANS, LLP, 2700 CAREW TOWER, 441 VINE STREET, CINCINNATI, OH, 45202, US

(21) Appl. No.: 09/986,260

(22) Filed: Nov. 8, 2001

Related U.S. Application Data
(63) Continuation of application No. PCT/US00/12877, filed on May 11, 2000.
(60) Provisional application No. 60/133,653, filed on May 11, 1999.

(51) Int. Cl. A61B 17/44

(52) U.S. Cl. 606/205; 606/151

(57) ABSTRACT

A clamping and fluid delivery device (50) for occluding a vessel (12) during a surgical procedure. Generally, the device (50) includes an internal core portion (52) having a distal end (52a) with a sealing surface and opposite side surfaces comprising sealing surfaces. The core portion (52) is inserted transversely into the vessel (12). Opposed, external clamping arms (62, 64) move together outside the vessel (12) and clamp the vessel (12) against the core portion (52). Padding (124) the sealing surfaces on opposite sides of the core portion (52), as well as on the distal end (52a) engage the internal walls (12a) of the vessel (12) and are opposed by padded clamping surfaces (120, 122) of the arms (62, 64). The core portion moves distally simultaneously with the clamping action of the arms to provide a distal seal. A bypass cannula (58) and cardioplegia cannula (60) fluidly couple the core portion (52) to deliver blood and cardioplegia fluid to opposite sides of the core portion (52).

The present application is a continuation of PCT Serial No. PCT/US00/12877 filed on May 11, 2000, now pending, which is based on provisional patent application Serial No. 60/133,653, filed May 11, 1999, now abandoned. The disclosures of each of these prior related applications are hereby fully incorporated by reference herein.

### FIELD OF THE INVENTION

The present invention generally relates to devices and methods for performing surgical procedures involving vessels such as the aorta and, more specifically, to clamping devices and methods particularly useful during cardiac bypass surgery and other cardiovascular procedures that involve temporarily arresting the heart.

### BACKGROUND OF THE INVENTION

During coronary artery bypass surgery, a surgeon bypasses an obstructed artery by shunting or redirecting flow from a large vessel, such as the aorta, to a part of the obstructed artery beyond the point of the obstruction. A variety of conduits or tubes may be used as grafts to carry this bypass blood flow. For example, the patient's own arteries and veins may be harvested or other artificial conduits may form the bypass.

During a typical bypass procedure, or any procedure which requires the heart to be stopped and placed on bypass such as Atrial Septal Defect (ASD) repair or valve repair, the heart and lungs of the patient are taken out of circulation by clamping the aorta and preventing retrograde flow of blood through the aortic valve into the left ventricle of the heart. Blood from the patient is redirected through a conventional heart-lung machine. More specifically, the surgeon places an aortic cross-clamp between the aortic valve and the first vessel of the aortic arch. While this procedure prevents blood from entering the heart, it also prevents oxygenated blood from perfusing the coronary arteries and thus places the heart into cardiac arrest in a controlled manner. The heart like other organs needs oxygenated blood to function when the blood supply is stopped to any organ it will begin to necrose or die. In order to stop the heart to repair defects without allowing the muscle to necrose, a liquid solution was developed called cardioplegia. Often, cardioplegia is administered between the aortic clamp and the heart through a separate infusion cannula to perfuse the arrested heart muscle. Cardioplegia is a liquid solution which usually contains potassium and is designed to maintain viability of the arrested heart muscle. If the aortic valve of the patient is functioning properly, the valve will seal against this flow of cardioplegia and allow the cardioplegia to enter the patient's coronary arteries surrounding the heart muscle. The area of the aorta between the aortic valve and the first arch vessel is a principle location for attaching proximal ends of the coronary bypass grafts.

During heart surgery, the risk of stroke increases with the age of the patient. At age 70 and above, the risk of stroke or brain disfunction during surgery approaches about 15%. The cause of this problem is not entirely clear, but increasing evidence suggests the occurrence of embolism, or movement of dislodged plaque, from the aorta into the arch vessels and on to the brain during heart surgery. Increasing evidence is implicating the aortic cross-clamp in the production of embolic debris.

Many devices have been developed to trap dislodged plaque debris before the debris is able to lodge in smaller arteries. For example, surgeons deploy nets and filters in the arteries and veins to trap and remove these emboli before they lodge in downstream arteries and vessels. Few devices or improvements have been directed to reducing the root cause of plaque dislodgment, there by reducing the risk of stroke. Plaque or calcium deposits can be hard and brittle. These deposits occur naturally throughout our life and form on the interior wall of many vessels, including the aorta. The vessel wall being flexible can be manipulated with a rigid clamp to close off the flow of blood by pinching the vessel between the flat jaws of an aortic cross clamp. Cross clamps have been designed to pinch and grip the slippery exterior surface of the vessel. However, if inflexible calcium deposits are present under the clamp the squeezing of the aortic tissue can these deposits the plaque.

Many types of clamps or intra-aortic balloon occlusion devices have been developed and some attempt to reduce the amount of debris created during aortic or other vessel clamping processes. As one measure used to reduce the amount of trauma during a clamping operation, soft jaws or pads have been used as exterior clamping surfaces. Unfortunately, even soft jaws will fold and severely compress the aorta. Therefore, dislodgment of plaque remains inevitable. When the aorta is compressed flat to form the necessary seal, the opposed aortic walls are parallel and therefore subject to moderate compressive forces. Along these walls, the load spreads out over a relatively large area with little elastic stretching of the tissue. However, where the aortic wall turns 180° at each of the compressed corners or folds and the aortic tissue at those corners is subject to massive compression and stretching forces. Plaque deposits at these corner locations, which may include hard or soft calcium deposits, can easily fracture and dislodge from the aortic wall.

Another type of clamping device, known generally as an intra-aortic balloon which is placed by a femoral cannula, inflates within the aorta to make full circumferential contact with the internal aortic wall surfaces. Often, the balloon will stretch the aortic wall as it occludes the blood flow. Like the external clamps, the balloon can dislodge hard or soft plaque from the aortic wall. The balloon dislodges plaque by extending and separating the soft flexible intimal lining of the aortic or vessel wall from the inflexible and often brittle plaque.

Due to various problems in the area of cardiovascular and vascular procedures in general, and especially bypass procedures, it would be desirable to provide clamping apparatus and methods designed to reduce the occurrence of embolism during surgery. In addition, with existing devices the surgeon must find physical space on the aorta to place three separate components, i.e., the cardioplegia cannula, the cross clamp and the aortic or bypass cannula. There is only a short distance of aortic vessel between the aortic valve and the first arch vessel in which to make the proximal graft connections, with three separate devices this valuable space is used up with inefficient components. Therefore, a consolidation of the hardware opens up space on the aortic vessel that can be used by the surgeon to place graft vessels. Therefore, a long felt and unrealized need must be addressed by efficiently combining components while reducing the trauma to the vessel wall.

Still further, the surgeon must make incisions in the aorta for both the aortic or bypass cannula and the cardioplegia cannula to gain access to the interior of the vessel. Each of these incision sites must be closed with sutures, such as purse string sutures when the cannulas are removed. In addition to the time it takes to place the sutures and install the cannulas each site is a prospective cause for leaks or tears in the aortic wall. As well as breaking plaque from the aorta while sealing/suturing these access sites. Further reduction of these risks is advantageous.

### SUMMARY OF THE INVENTION

In one aspect, the present invention provides a clamping device for occluding a vessel during a surgical procedure. The clamping device includes an internal core portion having a distal end with a sealing surface and opposite side surfaces comprising sealing surfaces adapted to be inserted transversely into the vessel through an incision in a wall of the vessel. An external clamping portion is provided and extends on the outside of the vessel. At least one of the core portion and the external clamping portion is movable with respect to the other to clamp the wall of the vessel between the sealing surfaces of the internal core portion and the external clamping portion. In accordance this aspect of the invention, the core portion is movable relative to the external clamping portion to adjust the length of the core portion within the vessel and to seat the distal sealing surface of the core portion against a portion of the vessel wall generally across from the incision. The distal sealing surface of the core portion is preferably rounded to further prevent fracturing plaque during a clamping procedure. The internal core portion and the external clamping portion may extend substantially perpendicularly across the vessel or at other desirable or necessary transverse angles across the vessel.

The external clamping portion is slidably movable along the core portion in the preferred embodiment but other types of movement may be used as well. The external clamping portion more specifically comprises first and second pivotally connected vessel engagement arms. These arms have clamping surfaces configured to receive and clamp the vessel and the core portion therebetween when brought together to a clamped position. At least one activating member and, more specifically, a pair of activating members couple the arms together in the form of a scissor linkage which moves the arms toward and away from one another and simultaneously moves the core portion relatively longitudinally with respect to the arms. In this manner, as the arms come together to a clamped position, the core portion moves distally farther into the vessel preferably until the distal sealing surface engages against the vessel wall generally across from the incision. In the preferred embodiment, the arms move relatively proximally back toward the core portion due to the action of a scissor linkage. The activating members preferably comprise manually-operable members configured to be squeezed together to facilitate this clamping and sealing action both inside and outside the vessel. Respective connecting elements on the first and second vessel engagement arms and the core portion connect the arms to the core portion, preferably in a removable manner. In the preferred embodiment, the connecting elements are C-shaped clips on the activating members which receive respective bosses on the core portion with a slight snap fit.

A ratchet mechanism is coupled with the vessel engagement arms and locks the arms in a fixed position relative to one another and further allows selective application of clamping pressure to the vessel. A ratchet release is also provided for providing release of the clamping pressure.

In another aspect of the invention, the core portion further includes at least one lumen for delivering a fluid from outside the vessel to within the vessel. More preferably, the core portion further includes two lumens for separately delivering blood and cardioplegia fluid to opposite sides of the core portion. These lumens may be separate cannulas extending into the core portion or integrally formed hollow spaces in the core portion or a combination of both as in the preferred embodiment. The internal core portion may further include a valve mechanism for selectively allowing fluid flow within the vessel between opposite sides of the internal core portion. As examples, the valve may include a slide member or a rotatable member used to regulate fluid flow.

The internal core portion preferably includes an inner portion having a first hardness and an outer portion having a second hardness less than the first hardness for contacting internal wall portions of the vessel. The outer portion includes the opposite side surfaces of the core portion which oppose sealing surfaces on the external clamping portion and include the sealing surface at the distal end of the core portion. For example, all opposed sealing surfaces of the core portion and the vessel engagement arms may be comprised of a soft polymeric material such as medical grade foam.

In another aspect of the invention, the distal ends of the vessel engagement arms are curved toward one another to present curved inner clamping surfaces configured to engage an opposing, rounded outer surface of the vessel across from the incision when the first and second vessel engagement arms are clamped in position on the vessel. The distal ends of the arms preferably include mating tips configured to engage one another in the clamped position. The mating tips preferably provide a self-centering action to longitudinally align the arms with each other in the clamped position. Since the distal mating tips mate together, the vessel cannot bulge outwardly at this location and leakage past the core portion in therefore prevented at the distal end of the core portion.

As another aspect of the invention, a sealing member is provided on the internal core portion and includes a sealing surface configured to seal against the vessel within the incision. Preferably, the sealing member is retained for movement along the internal core portion to provide an adjustment feature depending, for example, on the size of the vessel. The seal member is preferably retained on the core portion with a dynamic seal, such as an O-ring, allowing sliding movement. At least one seating surface extends on the sealing member for seating an adjustment member, such as a sliding tube, associated with a purse string suture applied around the incision. This feature allows the adjustment member or tube to be pushed against the sealing member to hold the sealing member in place within the incision. Typically, an adjustment tube associated with the purse string suture is clamped in position after tightening. This action will also fix the sealing member in its sealed position within the incision and inhibit fluid leakage from the vessel.

As another optional manner of providing longitudinal movement of the internal core portion, the core portion may be formed from a plurality of sections with at least one section being longitudinally adjustable relative to another to adjust the length of the core portion within the vessel.

A method of occluding the vessel in accordance with the invention generally includes making an incision in a wall of the vessel; inserting an internal core having a distal tip through the incision and into the vessel; moving the core into the vessel until the distal tip contacts an interior portion of the vessel wall generally across from the incision; placing an external clamp on an exterior side of the vessel wall; and moving at least one of the internal core and the external clamp toward the other to clamp the vessel wall between the external clamp and opposite sides of the internal core and between the distal tip of the core and the clamp.

The method can further include introducing fluid into the vessel through the internal core and, more specifically, introducing first and second fluids on opposite sides of the internal core. The moving step can further comprise moving the interior core relative to the clamp into the vessel. The method can further include engaging the wall of the vessel at the incision with a seal member disposed on the internal core. The seal member may be slid along the internal core and into the incision. After clamping, a valve mechanism may be operated to regulate fluid flow from one side of the core to the other.

These and other objects, advantages, and features of the invention will become more readily apparent to those of ordinary skill in the art upon review of the following detailed description of the preferred embodiments, taken in conjunction with the accompanying drawings.

### BRIEF DESCRIPTION OF THE DRAWINGS

FIG. 1 is a perspective view illustrating various elements of a conventional bypass procedure.

FIG. 2 is a cross sectional view of the aorta and cross clamp shown in FIG. 1 schematically illustrating full clamping of the aorta with the cross clamp.

FIG. 3 is a fragmented cross sectional view showing the aorta after the cross clamp has been released.

FIG. 4 is a perspective view showing the installation of a clamping and fluid introduction device constructed in accordance with the invention preparing to be introduced into the aorta.

FIG. 5 is a longitudinal cross sectional view of the clamping and fluid introduction device with a core portion thereof being inserted into the aorta.

FIG. 6 is a fragmented, cross sectional view of the clamping and fluid introduction device with the core portion fully inserted into the aorta.

FIG. 7 is a longitudinal cross sectional view of the clamping and fluid introduction device in the fully inserted position and showing the outer clamping members fully engaged with the outside of the aorta.

FIG. 8 is a partially fragmented, cross sectional view taken generally along line 8-8 of FIG. 7.

FIG. 9 is an exploded perspective view with the core portion and outer clamping portion longitudinally sectioned to show various details thereof.

FIG. 9A is a partially fragmented, perspective view of the distal tip of the core portion enlarged to show various details thereof.

FIG. 10 is a perspective view of an alternative embodiment illustrating a core portion with a slide valve.

FIG. 11 is a cross sectional view taken generally along line 11-11 of FIG. 10.

FIG. 12 is a perspective view illustrating another alternative core portion having a slide valve and a lengthwise adjustment feature.

FIG. 13 is a perspective view illustrating another alternative core portion having a rotatable butterfly valve.

FIG. 14 is a cross sectional view taken along line 14-14 of FIG. 13 and showing the clamping device and core portion applied to the aorta.

### DETAILED DESCRIPTION OF THE PREFERRED EMBODIMENTS

The present application is related to U.S. Provisional Application Serial No. 60/133,653, the disclosure of which is hereby incorporated by reference herein in its entirety.

In order to place a patient on a heart-lung machine to operate on a non-beating or arrested heart, the surgeon must gain access to the heart. Once the surgeon has opened the sternum and gained access to the heart, the patient must be placed on the heart-lung machine. One must first have a basic understanding of the circulatory system to understand the bypass operation. The inferior and superior vena cava bring non-oxygenated blood to the right atrium of the heart, which is essentially a holding compartment. The non-oxygenated blood is then transferred into the right ventricle of the heart, which is a pumping station. The non-oxygenated blood is pumped from the right ventricle to the lungs for oxygenation. Once the blood has been oxygenated in the lungs, it is returned to the heart into the left atrium. Like the right atrium, the left atrium is also a holding compartment. The oxygenated blood is then transferred into the left ventricle. The left ventricle is a high-pressure pump that pumps the oxygenated blood into the ascending aorta, which carries the blood throughout the body.

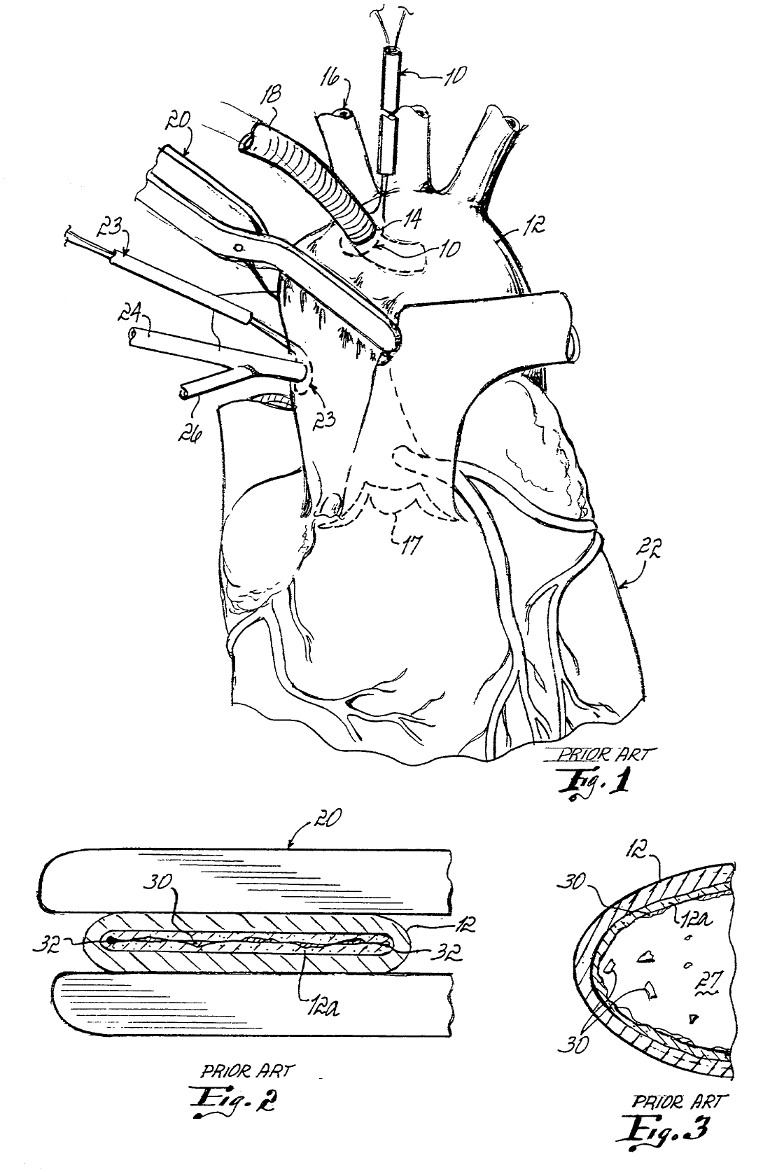

Referring to FIG. 1, in a conventional bypass procedure or any procedure that requires the surgeon to arrest the heart the surgeon will place a cannula (not shown) into the right atrium to divert the non-oxygenated blood flow from the body into the heart-lung machine. The surgeon must create access for the blood to return to the body once it has completed an oxygenation cycle in the heart-lung machine. FIG. 1 shows a purse string suture 10 in the ascending aorta 12 around the location of an incision 14 just proximal to the first arch vessel 16. Incision 14 is located between arch vessel 16 and aortic valve 17. The surgeon will make the incision 14 within the boundary of the purse string suture 10. A bypass cannula 18 is secured and sealed within incision 14 by purse string suture 10. This bypass cannula 18 returns oxygenated blood from the heart-lung machine to the patient. As further shown in FIG. 1, a conventional cross clamp 20 is used between the bypass cannula 18 and the patient's heart 22. A second purse string suture 23 and cannula 24 is installed between the cross clamp 20 and heart 22. Cannula 24 is used to administer cardioplegia to maintain the viability of heart 22 and includes a vent 26 used later for degassing the heart during start-up.

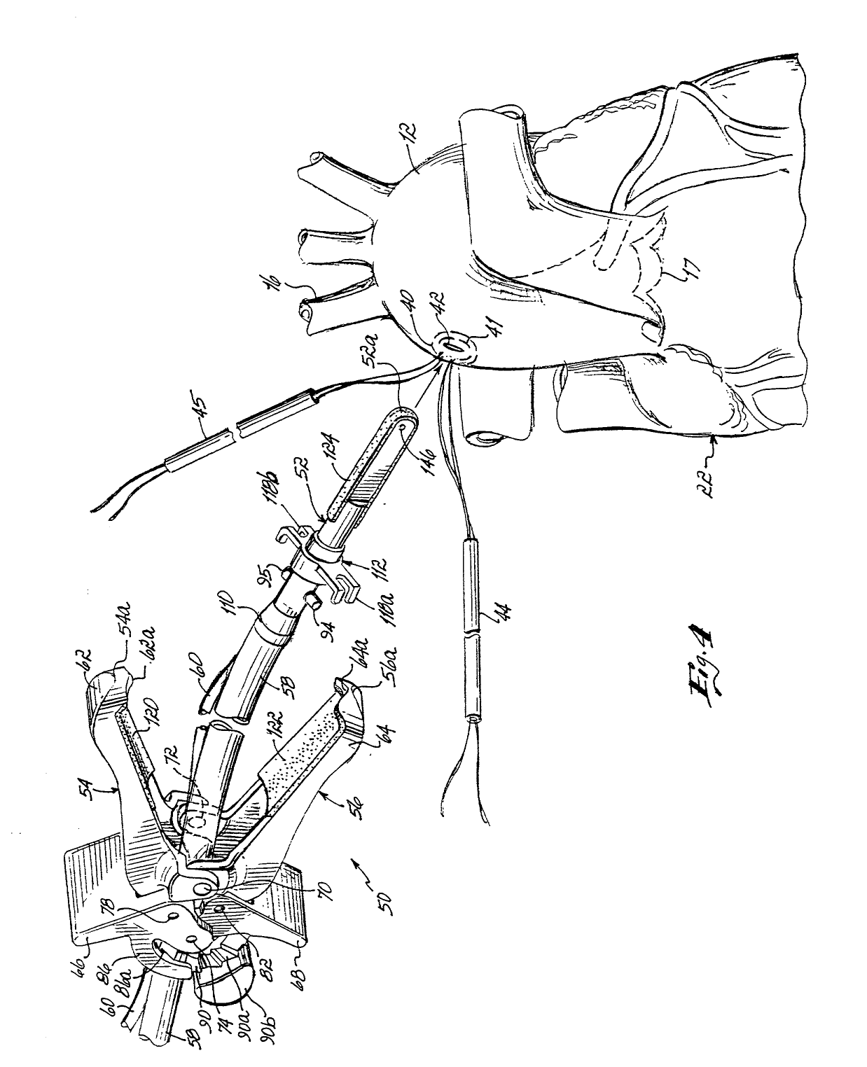

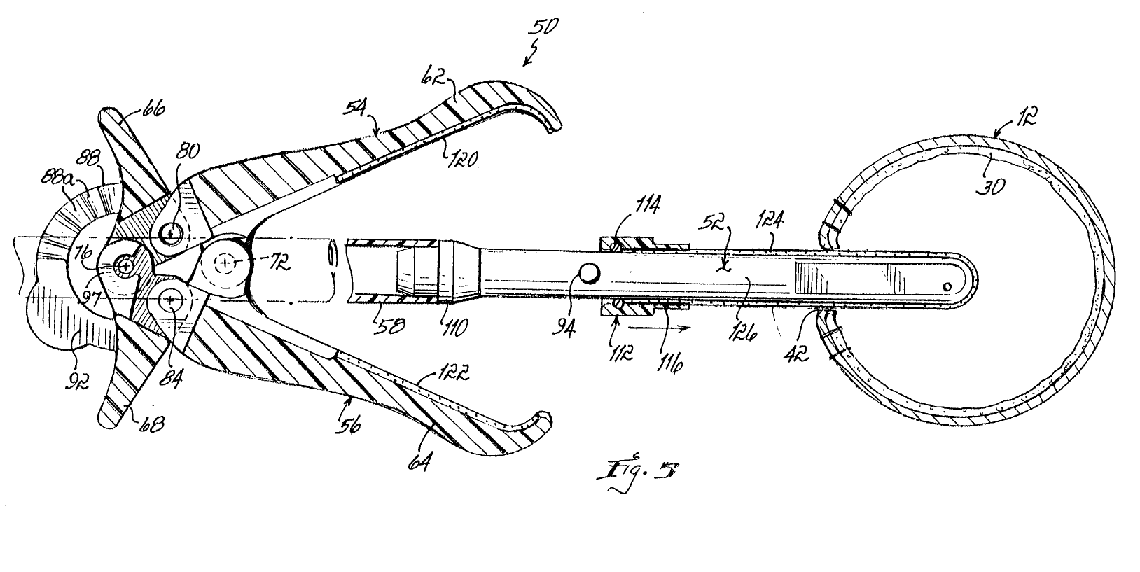

FIGS. 2 and 3 show how existing cross clamps 20 seal the aorta 12. These clamps 20 force the internal or intimal wall surfaces 12a of the aorta 12 together thereby preventing blood flow past clamp 20. Plaque 30 at the apex 32 of the fold cracks and separates from the intimal wall 12a of the aorta 12. As shown in FIG. 2, aorta 12 deforms and flattens in directions both parallel and transverse to its length. As FIG. 3 shows, once the aorta 12 is opened by removing clamp 20, dislodged, fractured plaque 30 is free to flow within the bloodstream 27 and potentially to lodge in a smaller downstream vessels and cause an embolism.

As FIG. 4 illustrates, practicing the present invention will preferably involve installing two purse string sutures 40, 41 about an incision 42 in preparation for placing a patient on a heart-lung machine. Two purse string sutures 40, 41 are used to provide a backup in case one fails. As further illustrated in FIG. 4, a two-part clamping device 50, constructed in accordance with a preferred embodiment of the invention, includes an elongate internal core portion 52 having a curved distal end 52a′ having respective curved distal ends 54a, 56a shaped in a generally complementary manner to distal end 52a of core portion 52 and external clamping pieces 54, 56. The purse string sutures 40, 41 are used to seal the aorta against core portion 52. When core portion 52 is removed, the purse string sutures 40, 41 are used to permanently seal incision 42. It will be appreciated that clamping device 50 preferably requires no additional incisions or larger incisions other than those typically made during bypass surgery. In this regard, and as detailed below, clamping device 50 can include a bypass input cannula 58 and a cardioplegia (CP) input cannula 60. Bypass input cannula 58 allows oxygenated blood to return to the patient's aorta 12 from the heart-lung machine (not shown), while cardioplegia may be administered to heart 22 on an opposite side of clamping device 50 through CP input cannula 60.

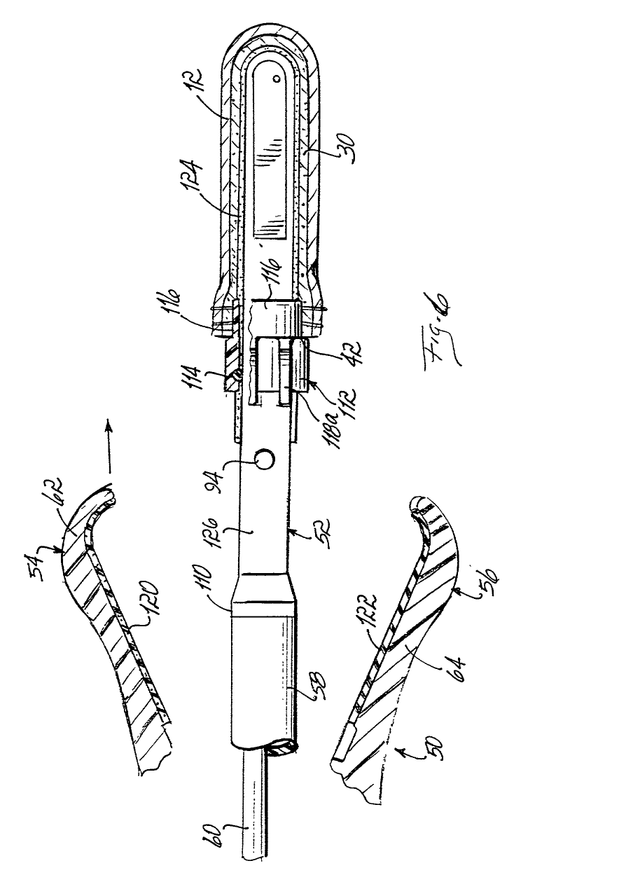

Referring now to FIGS. 4-9A, two-part clamping device 50 further includes a pair of vessel engagement arms 62, 64 each pivotally connected to one another, as well as pivotally connected to respective activating members 66, 68. More specifically, arms 62, 64 and activating members 66, 68 are pivotally coupled in a scissor-linkage arrangement. Arms 62, 64 are pivotally connected together at respective front pivots 70, 72 and activating members 66, 68 are pivotally connected together at respective rear pivots 74, 76. A pair of upper pivots 78, 80 pivotally connect arm 62 to activating member 66 and a pair of lower pivots 82, 84 pivotally connect arm 64 to activating member 68. For reasons to be discussed below, and as apparent by reviewing FIG. 5 in comparison to FIG. 7 respectively illustrating the open and closed positions of arms 62, 64, rear pivots 74, 76 will move in a forward direction toward the distal ends 54a, 56a of clamping pieces 54, 56 and arms 62, 64 when activating members 66, 68 are manually squeezed together by the surgeon. Respective ratchet members 86, 88 extend from activating member 66 and respective ratchet members 90, 92 extend in opposed relation to ratchet members 86, 88 from activating member 68. Ratchet members 86, 88, 90, 92 have respective ratchet teeth 86a, 88a, 90a, 92a which engage as shown in the figures to retain vessel engagement arms 62, 64 in the clamped position shown in FIG. 7. The distal tips 62a, 64a of arms 62, 64 are contoured as best shown in FIG. 4 to provide a self-centering action as arms 62, 64 are brought to the closed position shown in FIG. 7.

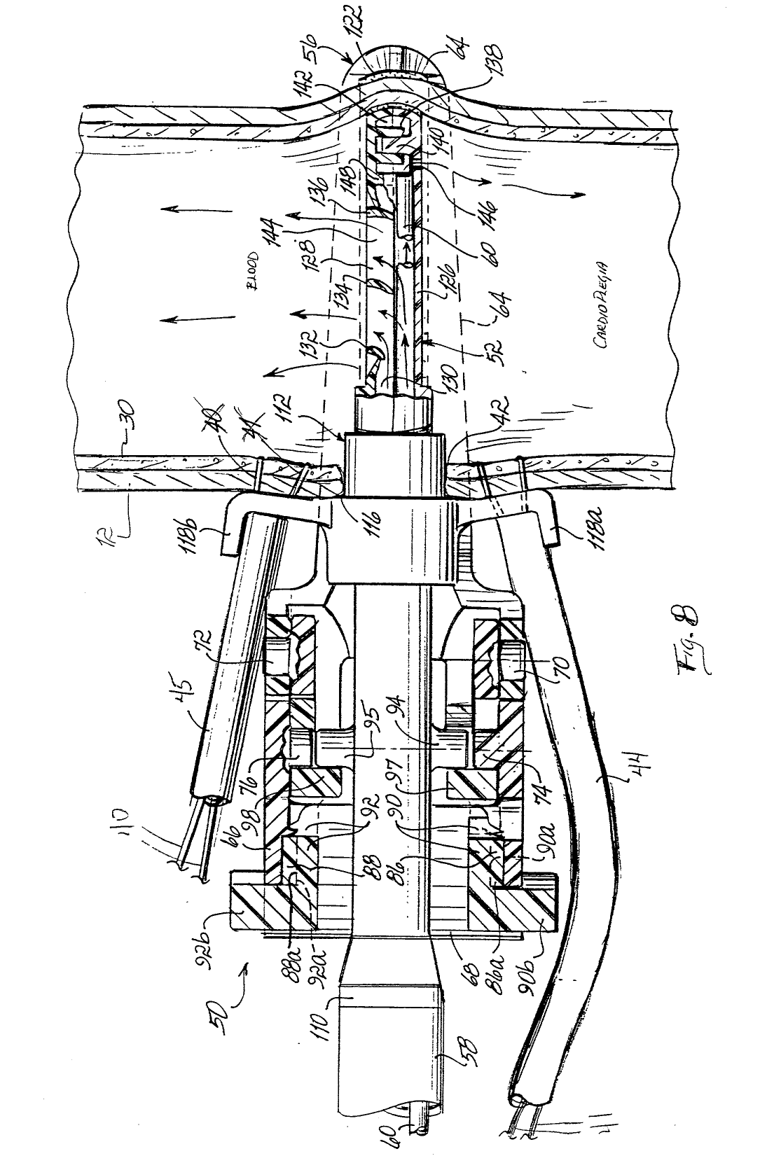

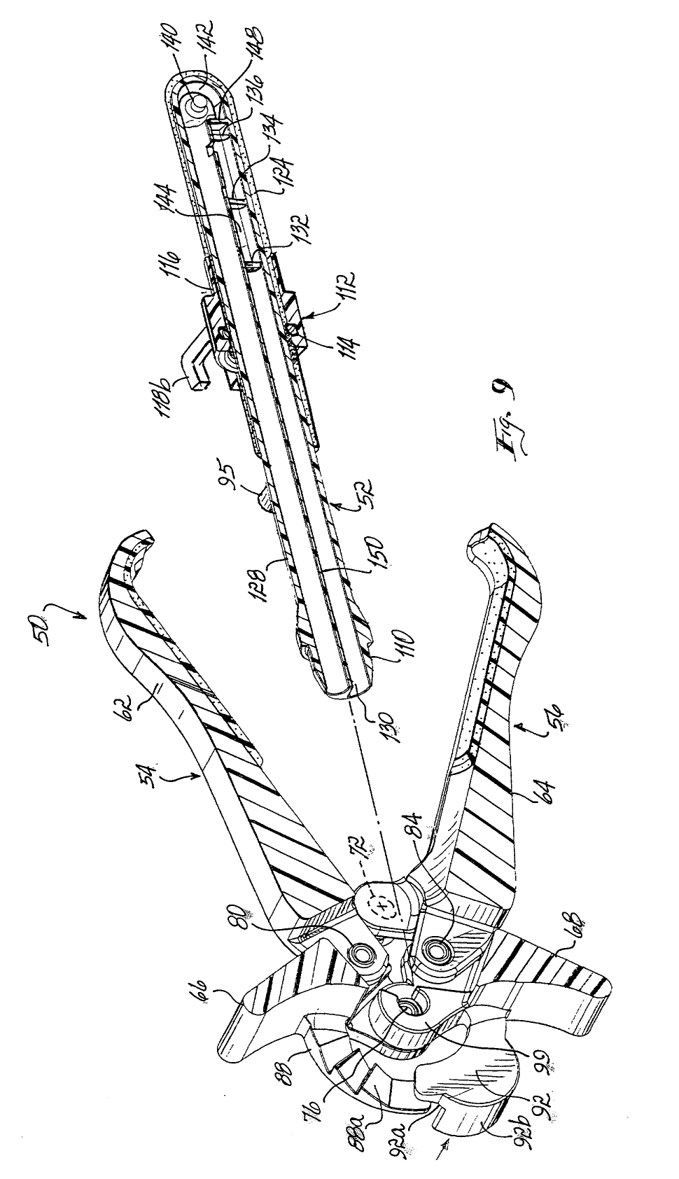

Core portion 52 includes oppositely extending bosses 94, 95 which are received with C-shaped clips or retainers 97, 99 preferably with a slight snap fit. A connector 110 is provided on core portion 52 for connecting bypass cannula 58. A seal member 112 is slidably retained on an outer surface of core portion 52 and slidably engages the outer surface of the core portion 52 with an O-ring seal 114. Seal member 112 includes a stepped-down portion 116 having an outer surface which sealingly engages aorta 12 at incision 42 to inhibit fluid leakage from aorta 12 as best shown in FIG. 8. Seal member 112 includes oppositely extending ears 118a, 118b having surfaces for seating respective tubes 44, 45 used to tighten purse string sutures 40, 41. Due to this feature, tubes 44, 48 may also be used to push against or retain seal member 112 within incision 42. For cushioning the clamping action of device 50 on aorta 12, a soft cover 120, 122 is provided on each arm 62, 64 and an opposed cover 124 is provided on core portion 52 extending in opposed relation to linings 120, 122 on opposite side surfaces of core portion 52 and further covering the distal tip of core portion 52 as shown in FIG. 8. These covers 120, 122, 124 may be formed of any suitable medical grade, relatively soft material such as foam, soft polymers, bladders, etc. In the preferred embodiment, covers 120, 122, 124 are formed from closed cell foam, while the remaining harder portions of core portion 52 and arms 62, 64 are molded from polycarbonate. Thus, cushioning is provided at all clamping contact points between the wall of aorta 12 and the respective inner surfaces of arms 62, 64 and outer surfaces of core portion 52. FIG. 9A shows that core portion 52 is preferably injection molded and assembled from first and second halves 126, 128 forming a hollow interior space 130. Ribs 132, 134, 136 are formed within hollow space 130 and function to evenly distribute blood flow from bypass cannula 58 through an opening 144 in core portion 52 and to prevent high blood flow impinging on and dislodging plaque 30. The distal end of core portion 52 includes a recess 138 and a mating boss 140 for connecting the two halves 126, 128 together. A distal chamber 142 is formed in core portion 52 and includes an opening 146 for delivering cardioplegia to the opposite side of core portion 52 relative to opening 144. A wall 148 separates distal chamber 142 from hollow space 130 and receives cardioplegia cannula 60 for the delivery of cardioplegia. A retainer 150 is formed in hollow space 130 and retains cardioplegia cannula 60 in place within hollow space 130.

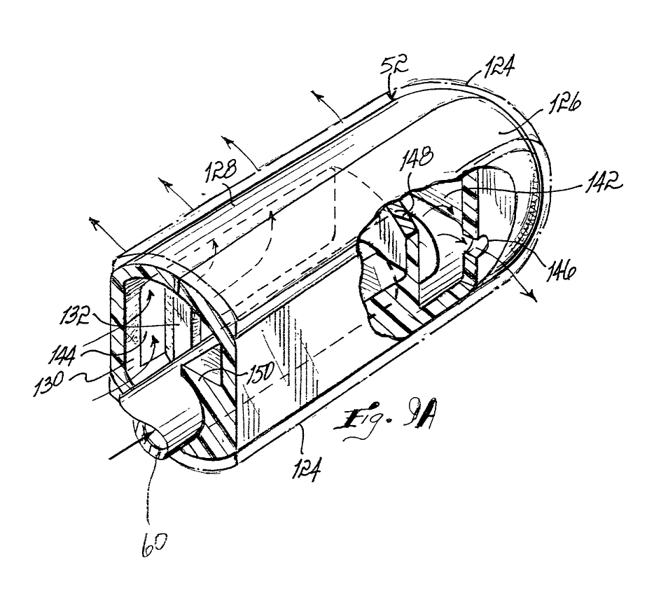

FIGS. 10 and 11 illustrate an alternative core portion 160 as another aspect of this invention. More specifically, core portion 160 includes a slide valve member 162 movable back-and-forth within core portion 160 as designated by arrow 163. Core portion 160 is usable in conjunction with, for example, clamping device 50 with the outer clamping pieces 54, 56 previously described, as shown in phantom lines. Slide valve member 162 includes an actuating member 164 at a proximal or outer position relative to the vessel being clamped and usable manually to push or pull slide valve member 162. A bypass cannula 166 and a cardioplegia cannula 168 are provided to respectively supply blood and cardioplegia fluid to first and second internal spaces 160a, 160b within core portion 160. Core portion 160 is used in generally the same manner to provide cardioplegia and blood to the aorta, as described above, but slide valve member 162 allows the surgeon to gradually restrict or increase blood flow as opposed to immediately starting or stopping blood flow. This is especially useful while placing a patient on a bypass or heart lung machine or taking the patient off of the bypass or heart-lung machine. Core portion 160 is preferably formed from an inner hard layer and an outer softer layer as previously described.

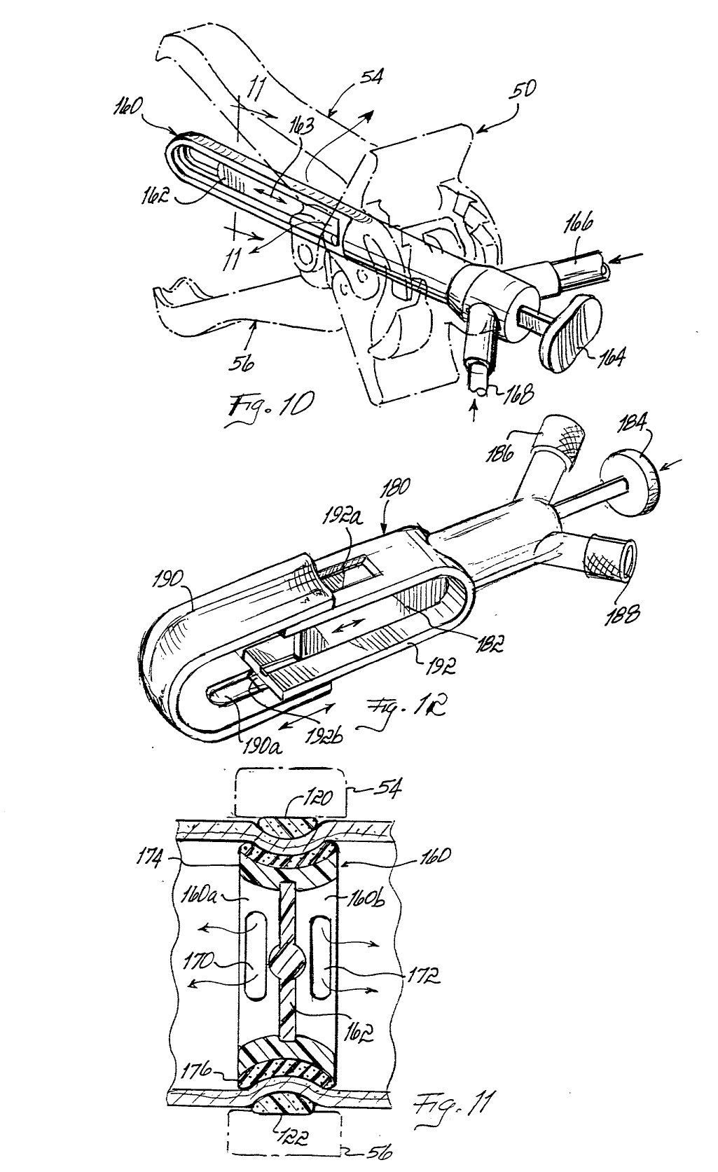

FIG. 12 illustrates another alternative core portion 180 including a slide valve member 182 and an actuating member 184 as generally described with respect to FIGS. 10 and 11. Core portion 180 may be used with clamping devices as generally described above, although the clamping device has been deleted for clarity. A bypass cannula 186 and a cardioplegia cannula 188 provide blood and cardioplegia fluid to internal sides of core portion 190 on either side of slide valve member 182 as in the embodiment of FIGS. 10 and 11. Also, the soft outer layer of core portion 180 has been removed for clarity. The main difference between core portion 180 and core portion 160 is that core portion 180 is comprised of a first section 190 and a second section 192. Sections 190, 192 are connected for lengthwise adjustment through recesses 190a, 192a and bosses 190b (only one shown). This allows core portion 180 to be length adjusted during insertion into a vessel, such as the aorta, and thereby tailored to the size of a particular patient's vessel or aorta.

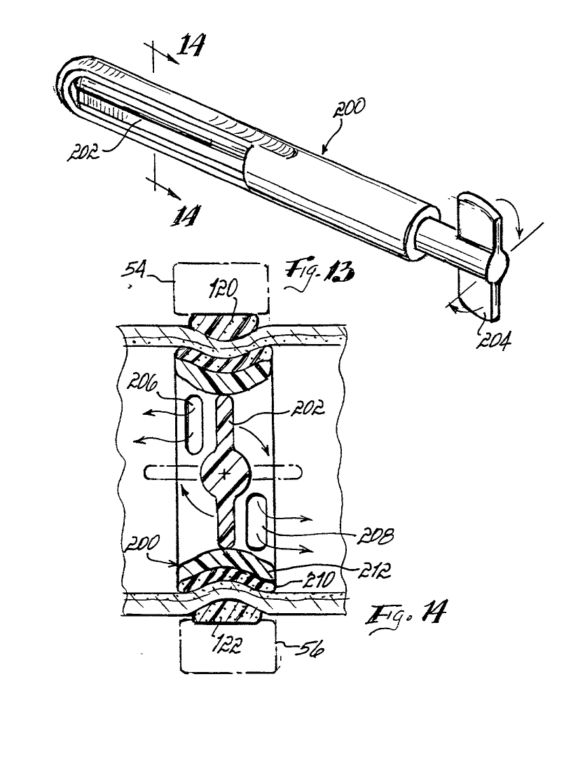

FIGS. 13 and 14 illustrate another alternative core portion 200 incorporating a butterfly valve member 202 which is rotatably actuated by an actuating member 204. Actuating member 204 may then be operated manually by a surgeon to gradually rotate butterfly valve member 202 between the closed position shown in solid lines in FIG. 14 and the full opened position shown in phantom lines. A blood inlet 206 and a cardioplegia fluid inlet 208 are provided in core portion 200 to allow inflow of blood and cardioplegia fluid on opposite sides of butterfly valve member 202 when in the closed position shown in FIG. 14. This blood may be introduced directly through actuating member 204 or through other suitable conduits coupled with inlets 206, 208. Core portion 200 again preferably comprises a hard inner layer 212 and a softer outer layer 210. FIG. 14 further illustrates clamping pieces 54, 56 operating in conjunction with core portion 200 similar to the previously described embodiments.

Operation

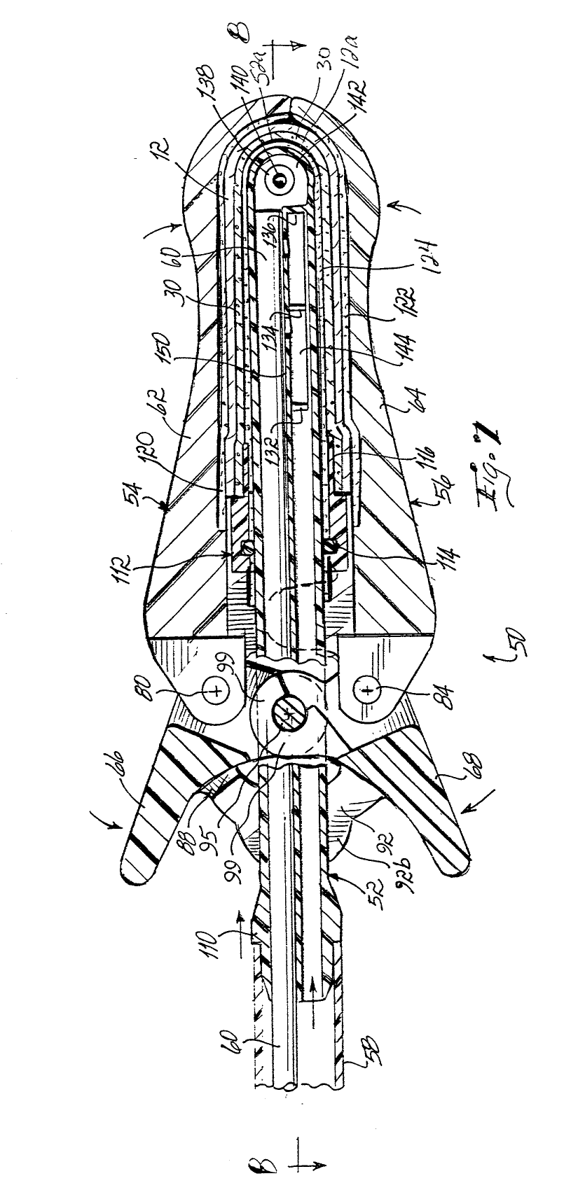

As shown in FIG. 4, an incision 42 is first made in aorta 12 between the first arch vessel 16 and aortic valve 17. First and second purse string sutures 44, 45 are placed about incision 42. Clamping and fluid delivery device 50 is prepared and held in the open or unclamped position shown with cannulas 58, 60 and core portion 52 extending between arms 62, 64. In this position, padding 124 on core portion 52 will oppose each padded surface 120, 122 of arms 62, 64. As shown in FIG. 5, core portion 52 is inserted into aorta 12 through incision 42 until, as shown in FIG. 6, seal member 112 engages aorta 12 within incision 42. Seal member 112 may be slid along core portion 52 until stepped portion 116 is fully contained within aorta 12. A seal is established between the outer surface of stepped portion 116 such that fluid is inhibited from leaking out of the interior of aorta 12 at this location. Also, O-ring 114 prevents fluid from leaking out of aorta 12 between core portion 52 and seal member 112.

FIG. 7 illustrates the fully clamped position of arms 62, 64 on the outside of aorta 12 and the position at which core portion 52 has been fully inserted into aorta 12 until a distal sealing surface 52a engages intimal wall 12a and any plaque 30, (illustrated schematically as a continuous layer, although typically formed as separate deposits, at that location). Padding 120, 122 along the inner surfaces of distal ends 54a, 56a directly opposes padding 124 at distal end 52a and lies on the outside of aorta 12 as shown. This forms a gradual curved transition area, as opposed to a sharp fold or apex, and inhibits the fracture of plaque in this area. As activating members 66, 68 are squeezed together from the position shown in FIG. 5 to the position shown in FIG. 7, C-shaped retainers 97, 99 move in a forward direction and, therefore, push bosses 94, 95 also in a distal direction. This direction, as viewed in FIG. 7 is to the right. At the same time, forward pivots 70, 72 move proximally and this pulls distal ends 54a, 54b toward distal end 52a for establishing a tight seal. This is due to the scissor linkage action and distal movement of rear pivots 74, 76 as activating members 66, 68 are squeezed together. As members 66, 68 are squeezed together, ratchet teeth 86a, 90a and 88a, 92a engage one another to lock arms 62, 64 in the clamped position. To release the arms, finger engagement portions 90b, 92b of ratchet members 90, 92 may be squeezed together to disengage the corresponding ratchet teeth 90a, 92a from respective ratchet teeth 86a, 88a.

While clamping and fluid delivery device 50 is in the clamped position, as shown in FIG. 8, adjustment tubes 44, 45 of purse string sutures 41, 40 are pushed in a distal direction and seat against ears 118a, 118b. This simultaneously tightens purse string sutures 40, 41 and pushes seal member 112 completely within incision 42 to the sealed, fully engaged position shown. At this stage, cardioplegia fluid may be administered through cannula 60 and bypass blood may be input through cannula 58 and into hollow space 130. Cardioplegia fluid will enter distal chamber 142 and exit through opening 146 to flow into aorta 12 on one side of core portion 52, while blood will flow through opening 144 into the opposite side of aorta 12 to circulate through the patient's body. Ribs 132, 134, 136 will prevent the high pressure blood flow from impinging with great force on the inner walls of aorta 12 and, therefore, functions as another manner of reducing separation of plaque 30 from the inner wall of aorta 12.

When one of the embodiments shown in FIGS. 10-14 is utilized, core portion 160, 190 or 200 may be used to regulate blood flow to and from the heart, especially during the procedures of placing the patient on a heart-lung machine and taking the patient off of the heart-lung machine.

While the present invention has been illustrated by a description of a preferred embodiment and while this embodiment has been described in some detail, it is not the intention of the Applicants to restrict or in any way limit the scope of the appended claims to such detail. Additional advantages and modifications will readily appear to those skilled in the art. The various features of the invention may be used alone or in numerous combinations depending on the needs and preferences of the user. This has been a description of the present invention, along with the preferred methods of practicing the present invention as currently known. However, the invention itself should only be defined by the appended claims, wherein we claim:

1. A clamping device for occluding a vessel during a surgical procedure, the clamping device comprising:
an internal core portion having a distal end with a sealing surface and opposite side surfaces comprising sealing surfaces adapted to be inserted transversely into the vessel through an incision in a wall of the vessel, and
an external clamping portion adapted to extend on the outside of the vessel, at least one of said core portion and said external clamping portion being movable with respect to the other to clamp the wall of the vessel between said internal core portion and said external clamping portion, said core portion being movable relative to said external clamping portion to adjust the length of said core portion within the vessel and to seat the sealing surface at the distal end against the vessel wall generally across from the incision.

2. The clamping device of claim 1, wherein said external clamping portion is slidably movable along said core portion.

3. The clamping device of claim 1, wherein said external clamping portion further comprises first and second pivotally connected vessel engagement arms, said vessel engagement arms having clamping surfaces configured to receive and clamp the vessel and said core portion therebetween when brought together to a clamped position.

4. The clamping device of claim 3 further comprising:
respective connecting elements on said first and second vessel engagement arms for connecting said arms to said core portion, and
an activating member coupled to one of said first and second arms and operable to move said one arm toward the other and to move said core portion longitudinally between said arms.

5. The clamping device of claim 4 further comprising:
a pair of said activating members coupled to said arms in the form of a scissor linkage which simultaneously moves said arms toward and away from one another and moves said core portion longitudinally with respect to said arms to facilitate seating the sealing surface of said distal end against the vessel wall.

6. The clamping device of claim 5, wherein said activating members further comprise manually operable members configured to be squeezed together to move said arms together with a clamping motion on the outside of the vessel.

7. The clamping device of claim 6 further comprising a ratchet mechanism coupled with said arms for locking said arms in a fixed position relative to one another and allowing selective application of clamping pressure to said vessel.

8. The clamping device of claim 1 further comprising a ratchet mechanism coupled with said clamping portion for locking said clamping portion in a fixed position relative to one another and allowing selective application of pressure to said vessel.

9. The clamping device of claim 1, wherein said core portion further includes at least one lumen for delivering a fluid from outside the vessel to within the vessel.

10. The clamping device of claim 1, wherein said core portion further includes two lumens for separately delivering blood and cardioplegia fluid to opposite sides of said core portion.

11. The clamping device of claim 1, wherein the internal core portion further includes a valve mechanism for selectively allowing fluid flow within the vessel between opposite sides of said internal core portion.

12. The clamping device of claim 1, wherein the internal core portion includes an inner portion having a first hardness and an outer portion having a second hardness less than the first hardness for contacting internal wall portions of the vessel, said outer portion including said opposite side surfaces and said sealing surface at said distal end.

13. The clamping device of claim 12, wherein said clamping portion includes outer portions having a first hardness and inner portions having a second hardness less than said first hardness, said inner portions of said clamping portion adapted to contact an outer surface of the vessel wall in opposed relation to the respective opposite side surfaces of said inner core portion.

14. The clamping device of claim 1, wherein said clamping portion further comprises:
first and second opposed vessel engagement arms having respective distal ends, said distal ends being curved toward one another to present curved inner surfaces configured to engage an opposite outer surface of said vessel from said incision when said first and second vessel engagement arms are in a clamped position on the vessel.

15. The clamping device of claim 14, wherein said distal ends include mating tips configured to engage one another in the clamped position.

16. The clamping device of claim 15, wherein said mating tips provide a self-centering action to longitudinally align said arms with each other in the clamped position.

17. The clamping device of claim 1, further comprising a sealing member retained for movement along said core portion and having an outer sealing surface configured to extend within the incision and seal against the vessel, said sealing member further including an inner sealing surface sealing against said core portion.

18. The clamping device of claim 17, further comprising a seating surface extending on said sealing member for seating an adjustment member associated with a purse string suture applied around the incision.

19. The clamping device of claim 1, wherein said core portion includes a plurality of sections and at least one section is longitudinally adjustable relative to another to adjust the length of said core portion with in the vessel.

20. A clamping and fluid delivery device for occluding a vessel during a surgical procedure and for simultaneously delivering at least one fluid to said vessel, the clamping and fluid delivery device comprising:
first and second opposed vessel engagement arms having opposed sealing surfaces adapted to extend on the outside of the vessel and movable between clamped and unclamped positions, and
an internal core portion having a distal end with a sealing surface and opposite side surfaces comprising sealing surfaces, said internal core portion adapted to be inserted transversely into the vessel through an incision in a wall of the vessel and further configured to be received between said opposed vessel engagement arms when in the clamped position such that each of said sealing surfaces of said core portion sealingly engages the wall of the vessel to inhibit fluid flow across the core portion within the vessel and each of said sealing surfaces of said core portion is opposed by a respective sealing surface of one of said arms bearing against the outer surface of the vessel wall.

21. The clamping and fluid delivery device of claim 20, wherein said external clamping portion is slidably movable along said core portion.

22. The clamping and fluid delivery device of claim 20, wherein said first and second vessel engagement arms are pivotally coupled together.

23. The clamping and fluid delivery device of claim 20 further comprising:
respective connecting elements on said first and second vessel engagement arms for connecting said arms to said core portion, and
an activating member coupled to one of said first and second arms and operable to move said one arm toward the other and to move said core portion longitudinally between said arms.

24. The clamping and fluid delivery device of claim 23 further comprising:
a pair of said activating members coupled to said arms in the form of a scissor linkage which simultaneously moves said arms toward and away from one another and moves said core portion longitudinally with respect to said arms to facilitate seating the sealing surface of said distal end against the vessel wall.

25. The clamping and fluid delivery device of claim 24, wherein said activating members further comprise manually operable members configured to be squeezed together to move said arms together with a clamping motion on the outside of the vessel.

26. The clamping and fluid delivery device of claim 25 further comprising a ratchet mechanism coupled with said arms for locking said arms in a clamping position relative to said core portion and allowing selective application of clamping pressure to said vessel.

27. The clamping and fluid delivery device of claim 20 further comprising a ratchet mechanism coupled with said clamping portion for locking said clamping portion in a clamping position relative to said core portion and allowing selective application of pressure to said vessel.

28. The clamping and fluid delivery device of claim 20, wherein said core portion further includes at least one lumen for delivering a fluid from outside the vessel to within the vessel.

29. The clamping and fluid delivery device of claim 20, wherein said core portion further includes two lumens for separately delivering blood and cardioplegia fluid to opposite sides of said core portion.

30. The clamping and fluid delivery device of claim 20, wherein the internal core portion further includes a valve mechanism for selectively allowing fluid flow within the vessel between opposite sides of said internal core portion.

31. The clamping and fluid delivery device of claim 20, wherein the internal core portion includes an inner portion having a first hardness and an outer portion having a second hardness less than the first hardness for contacting internal wall portions of the vessel, said outer portion including said opposite side surfaces and said sealing surface at said distal end.

32. The clamping and fluid delivery device of claim 20, wherein each arm includes a supporting portion having an inner clamping surface formed of softer material than said supporting portion.

33. The clamping and fluid delivery device of claim 20, wherein said clamping portion further comprises:
first and second opposed vessel engagement arms having respective distal ends, said distal ends being curved toward one another to present curved inner surfaces configured to engage an opposite outer surface of said vessel from said incision when said first and second vessel engagement arms are in a clamped position on the vessel.

34. The clamping and fluid delivery device of claim 33, wherein said distal ends include mating tips configured to engage one another in the clamped position.

35. The clamping and fluid delivery device of claim 34, wherein said mating tips provide a self-centering action to longitudinally align said arms with each other in the clamped position.

36. The clamping and fluid delivery device of claim 20, further comprising a sealing member retained for movement along said core portion and having an outer sealing surface configured to extend within the incision and seal against the vessel, said sealing member further including an inner sealing surface sealing against said core portion.

37. The clamping and fluid delivery device of claim 36, further comprising a seating surface extending on said sealing member for seating an adjustment member associated with a purse string suture applied around the incision.

38. The clamping and fluid delivery device of claim 20, wherein said core portion includes a plurality of sections and at least one section is longitudinally adjustable relative to another to adjust the length of said core portion within the vessel.

39. The clamping and fluid delivery device of claim 20, wherein said lumen includes flow diverting structure for distributing the outflow of fluid along a predetermined length of said core portion.

40. The clamping and fluid delivery device of claim 20, wherein said lumen further comprises a hollow space within said core portion for receiving blood, and said hollow space further contains a second lumen for carrying cardioplegia fluid, said second lumen opening to an opposite side of said core portion relative to said hollow space.

41. The clamping and fluid delivery device of claim 40, wherein said second lumen is contained in a cannula carried within said hollow space and opening to a chamber within said core portion, said chamber being sealed from said hollow space and opening to said opposite side of said core portion.

42. A clamping device for occluding a vessel during a surgical procedure, the clamping device comprising:
an internal core portion adapted to be inserted transversely into the vessel through an incision in a wall of the vessel,
an external clamping portion adapted to extend on the outside surface of the vessel, at least one of the core portion and the external clamping portion being movable with respect to the other to clamp the wall of the vessel between the core portion and the external clamping portion, and
a seal member disposed for movement along said core portion and configured to seat against the vessel within the incision to inhibit fluid leakage from the vessel.

43. The clamping device of claim 42, wherein said seal member is coupled for sliding movement lengthwise along said core portion to allow movement toward and away from the incision.

44. The clamping device of claim 43 further comprising a dynamic seal disposed between said seal member and said core portion to allow sliding lengthwise movement of said seal member along said core portion.

45. The clamping device of claim 42 further comprising at least one seating surface extending on said seal member for engaging an adjustment member of a purse string suture applied around the incision and allowing said adjustment member to push against said seal member and hold said seal member in sealing engagement within the incision.

46. A clamping device for occluding a vessel during a surgical procedure, the clamping device comprising:
an internal core portion having a rounded distal sealing end adapted to be inserted transversely into the vessel through an incision in a wall of the vessel and to sealingly engage a portion of the wall generally across from the incision, and
an external clamping portion including first and second opposed vessel engagement arms adapted to extend on the outside of the vessel, at least one of said arms being movable toward the other into a clamped position to clamp the wall of the vessel between said internal core portion and said arms, said arms further including distal tips having internal clamping surfaces curved to generally follow the curvature of the rounded distal sealing end of said internal core portion when in the clamped position thereby effectively clamping the vessel while inhibiting the loosening or breakage of plaque retained on internal surfaces of the wall of the vessel.

47. The clamping device of claim 46, wherein said external clamping portion is slidably adjustable along said core portion.

48. The clamping device of claim 46, wherein said distal tips of said arms have mating tips that engage one another in the clamped position.

49. The clamping device of claim 48, wherein said complementary contours provide a self-centering action to longitudinally align said arms with each other in the clamped position.

50. The clamping device of claim 46, wherein the internal core portion further includes a valve mechanism for selectively allowing fluid flow within the vessel between opposite sides of said internal core portion.

51. The clamping device of claim 46, wherein the internal core portion includes an inner portion having a first hardness and an outer portion having a second hardness less than the first hardness for contacting internal wall portions of the vessel, said outer portion including opposite side surfaces and said distal sealing end.

52. The clamping device of claim 46 further comprising at least one fluid input for directing fluid into the vessel through said incision.

53. A clamping device for occluding a vessel during a surgical procedure, the clamping device comprising:
an internal core portion adapted to be inserted transversely into the vessel through an incision in a wall of the vessel,
an external clamping portion adapted to extend on the outside of the vessel, at least one of said core portion and said external clamping portion being movable with respect to the other to clamp the wall of the vessel between said internal core portion and said external clamping portion, and
a valve mechanism carried by said internal core portion for disposition within said vessel and for selectively allowing fluid flow within the vessel between opposite sides of said internal core portion.

54. The clamping device of claim 53, wherein said valve mechanism includes a slide valve member carried for sliding movement to selectively expose and block an opening in said internal core portion.

55. The clamping device of claim 53, wherein said valve mechanism includes a valve member carried for rotatable movement within said internal core portion to selectively expose and block an opening in said internal core portion.

56. A method of occluding a vessel in a patient undergoing a surgical procedure, the method comprising:
making an incision in a wall of the vessel,
inserting an internal core having a distal tip through the incision and into the vessel,
moving the core into the vessel until the distal tip contacts an interior portion of the vessel wall generally across from the incision,
placing an external clamp on an exterior side of the vessel wall, and
moving at least one of the internal core and the external clamp toward the other to clamp the vessel wall between the external clamp and opposite sides of the internal core and between the distal tip of the core and the clamp.

57. The method of claim 56 further comprising:
introducing a fluid into the vessel through the internal core.

58. The method of claim 57 further comprising:
introducing a first fluid on one of the opposite sides of the internal core, and
introducing a second fluid on the other of the opposite sides of the internal core.

59. The method of claim 56, wherein the moving steps further comprise:
relatively moving the distal tip of the internal core into contact with the interior portion of the vessel simultaneously with moving at least one of the internal core and the external clamp toward the other to clamp the vessel.

60. The method of claim 59 further comprising:
engaging the wall of the vessel at the incision with a seal member disposed on the internal core.

61. The method of claim 60 further comprising:
sliding the seal member along the internal core and into the incision.

62. The method of claim 56 wherein the internal core further includes a valve mechanism and the method further comprises:
operating the valve mechanism to regulate fluid flow from one of the opposite sides to the other.
